(19) United States
(12) Patent Application Publication
(10) Pub. No.: US 2021/0198197 A1
(43) Pub. Date: Jul. 1, 2021

(54) METHOD FOR SYNTHESIZING INDOMETHACIN AND ANALOGUE THEREOF

(71) Applicant: Huazhong University of Science and Technology, Wuhan (CN)

(72) Inventors: Zhilong CHEN, Wuhan (CN); Helin LU, Wuhan (CN)

(21) Appl. No.: 17/110,590

(22) Filed: Dec. 3, 2020

(30) Foreign Application Priority Data
Dec. 3, 2019 (CN) 201911219492.2

(51) Int. Cl. C07D 209/26; C07D 405/06; B01J 31/22; B01J 27/232; B01J 31/02; B01J 27/128; B01J 31/30

(52) U.S. Cl. CPC C07D 209/26; C07D 405/06; B01J 31/2226; B01J 27/232; B01J 31/0275; B01J 2231/49; B01J 31/30; B01J 2531/824; B01J 2531/007; B01J 2531/008; B01J 2231/44; B01J 27/128

(57) ABSTRACT

The present disclosure belongs to the technical field of indomethacin synthesis, and discloses a method for synthesizing an indomethacin and an analogue thereof. The method for synthesizing an indomethacin and an analogue thereof includes steps of: introducing an alkyl group, an aromatic ring or a heteroaromatic ring directly at the C2 position of indole, a carboxylic acid fragment at the C3 position of the indole, and an aroyl group at the N1 position of the indole through palladium-catalyzed reactions. The present disclosure solves a problem: most of the existing indomethacin synthesis methods are achieved by construction of an indole ring and modification; simple structural changes of an indomethacin molecule based on this synthetic strategy often require de novo synthesis; the late modification and structure-activity relationship study of the indomethacin molecule have lengthy synthetic steps.

### CROSS REFERENCE TO RELATED APPLICATIONS

The present application claims the benefit of priority under 35 USC 119 to Chinese patent application 201911219492.2, filed Dec. 3, 2019, the contents of which are incorporated herein by reference.

### TECHNICAL FIELD

The present disclosure belongs to the technical field of indomethacin synthesis, in particular to a method for synthesizing an indomethacin and an analogue thereof.

### BACKGROUND

Indomethacin is a nonsteroidal anti-inflammatory drug (NSAID) that regulates a series of cell behaviors by inhibiting cyclooxygenase (COX) and thereby regulates the synthesis of downstream prostaglandin (PG) thereof. It is clinically commonly used for anti-inflammation and analgesia. In recent years, some analogues of indomethacin have been found to have a certain ability to reverse tumor drug resistance (J. Med. Chem. 2012, 55, 8152-63). COX is found to be highly expressed in many tumor cells, so COX inhibitors are also used in adjuvant therapy for tumors in clinical studies (Br. J. Cancer. 2011, 105, 452-459), and plays an important role in tumor immune escape (Cancer. Immunol. Res. 2017, 5, 695-709; Cell. 2015, 162, 1257-1270). Although a plurality of NSAIDs have been developed, there is still much room for more effective anti-inflammatory drugs with less side effects. It is an important aspect of new drug research and development to improve the targeted selectivity of drugs, drug safety and other properties through the structural modification of old drugs, and to develop new uses. Therefore, development of indomethacin and rapid construction of analogues thereof can provide potentially powerful tools for new drug research and development.

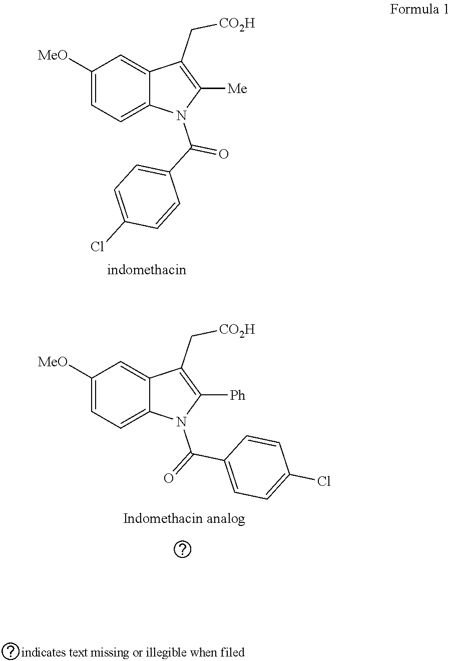 [SMILES: COc1ccc2c(c1)c(CC(=O)O)c(C)n2C(=O)c1ccc(Cl)cc1 ; COc1ccc2c(c1)c(CC(=O)O)c(-c1ccccc1)n2C(=O)c1ccc(Cl)cc1]

The conventional indomethacin synthesis usually depends on the construction of an indole ring. For example, Merck has developed an indomethacin synthesis method based on Fishcer indole synthesis (Org. Lett. 2004, 6, 79-82). This method is used to synthesize indomethacin per se. Synthesis of analogues thereof requires different raw materials, and a plurality of these raw materials, such as phenylhydrazine derivatives, are not commercially available or require a plurality of preparation steps (Formula 2).

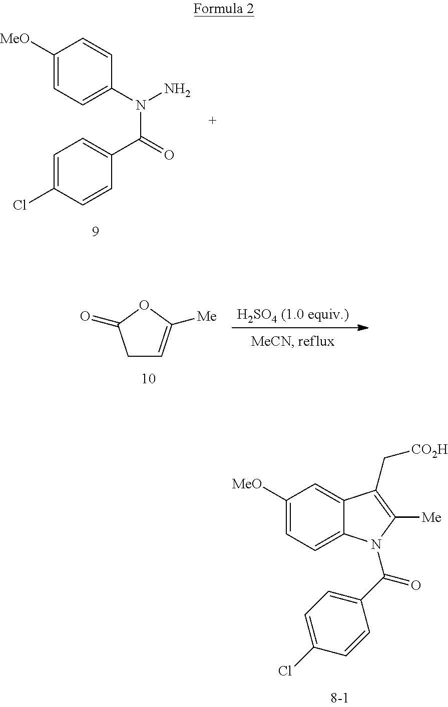

Transition metal-catalyzed indole synthesis is widely used in organic synthesis, and frequently used in the indomethacin synthesis. For example, Professor Kanae Mukai from Kanazawa University, Japan reported a method for synthesizing indomethacin skeleton using o-iodoaniline 10 and allene 11 as raw materials (Org. Lett. 2005, 7, 5793-5796). However, this method needs to use a relatively difficult-to-prepared allene 11 as a raw material, so the cost required for the synthesis of indomethacin above the gram level is relatively high (Formula 3).

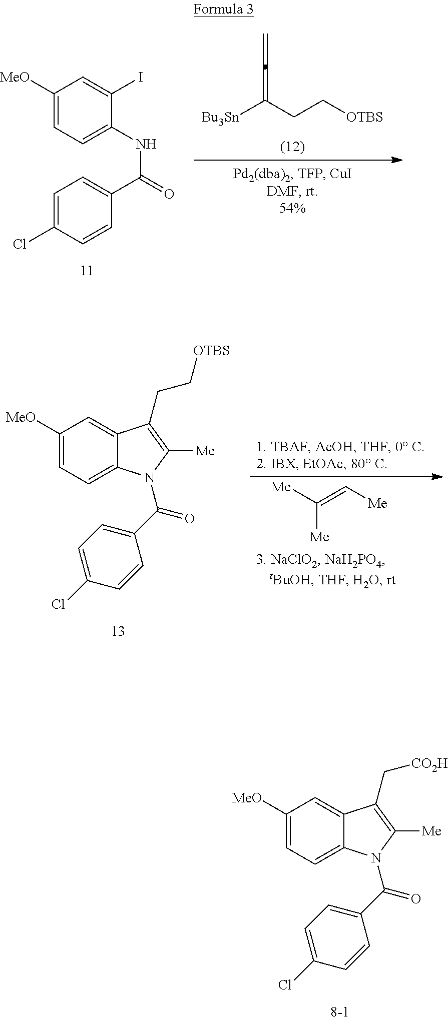

Researcher MA Shengming from Shanghai Institute of Organic Chemistry reported a method for synthesizing indole using o-iodoaniline 13 and propargyl bromide 14 as raw materials, and applied the method in the gram-grade synthesis of indomethacin (Org. Lett. 2013, 15, 2782-2785; Chinese Patent Publication No. CN103012241A, Formula 4).

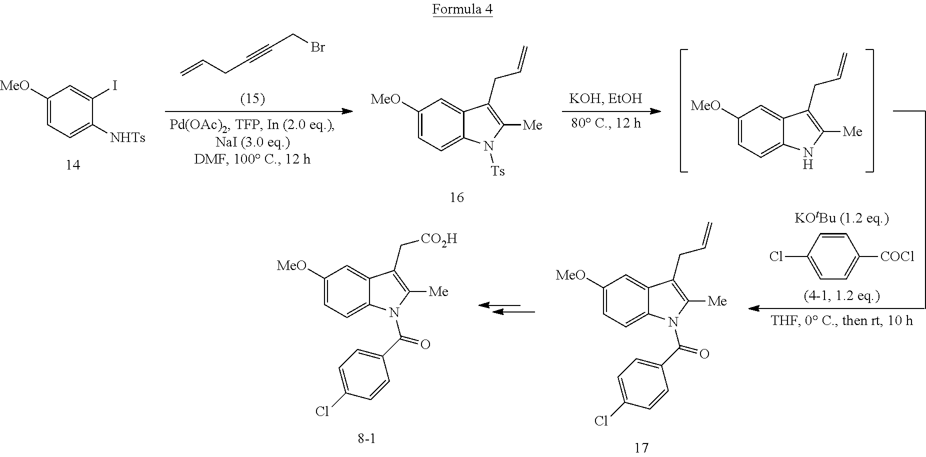

Professor ZHANG Chi from Nankai University reported another method for synthesizing indomethacin and analogues thereof. O-vinylaniline 18 as a raw material was coupled to indomethacin nucleus 19 by excessive iodine oxidation, followed by introducing dimethyl malonate at the C3 position of indole to obtain compound 20, decarboxylation and hydrolysis to obtain indomethacin (Chinese Patent Publication No. CN 108752257 A, Formula 5).

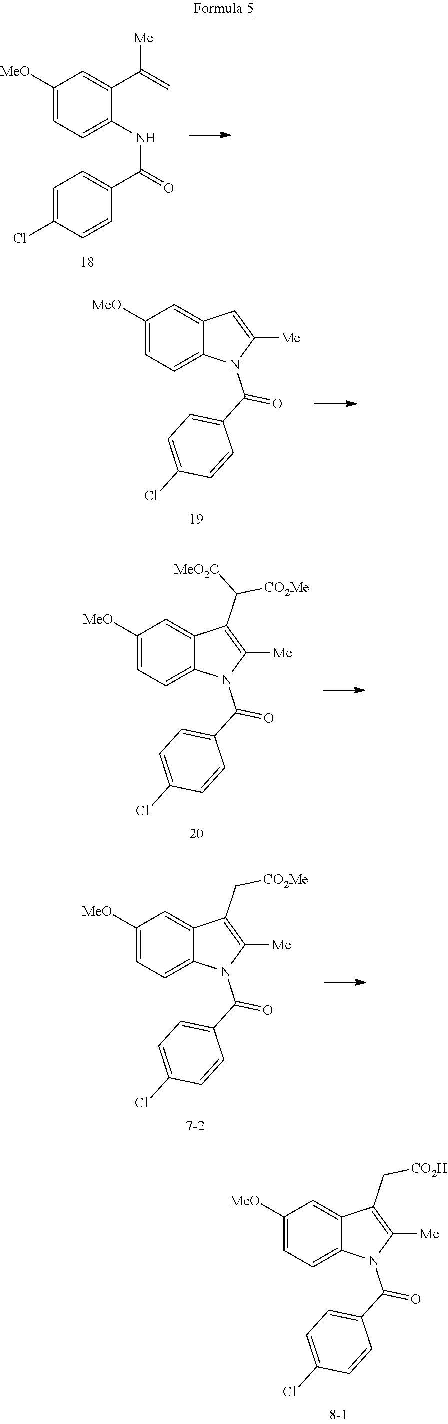 [SMILES: C=C(C)c1cc(OC)ccc1NC(=O)c1ccc(Cl)cc1 ; COc1ccc2c(c1)cc(C)n2C(=O)c1ccc(Cl)cc1 ; COC(=O)C(C(=O)OC)c1c(C)n(C(=O)c2ccc(Cl)cc2)c2ccc(OC)cc12 ; COC(=O)Cc1c(C)n(C(=O)c2ccc(Cl)cc2)c2ccc(OC)cc12 ; COc1ccc2c(c1)c(CC(=O)O)c(C)n2C(=O)c1ccc(Cl)cc1]

The development of the above synthetic methods provides an excellent tool for indomethacin synthesis, but considering the potential of indomethacin as a COX inhibitor in other disease areas, synthesis of indomethacin analogues can provide a strong support for the development of the corresponding drugs. However, in previous studies, synthesis of such indomethacin analogues often required lengthy synthetic steps, which were not conducive to rapid structure-activity relationship studies. For example, Shuto reported a method for synthesizing an indomethacin and an analogue thereof starting from an o-vinylaniline compound 21, but in this method, compound 5 is merely required for the construction of indole ring sometimes. At the same time, the method is difficult to provide more indomethacin analogues due to factors such as functional group tolerance and substrate limitations, which limits further structural modification (J. Med. Chem. 2012, 55, 8152). Therefore, it is of great significance to develop a more concise and efficient method for synthesizing indomethacin and analogues thereof (Formula 6).

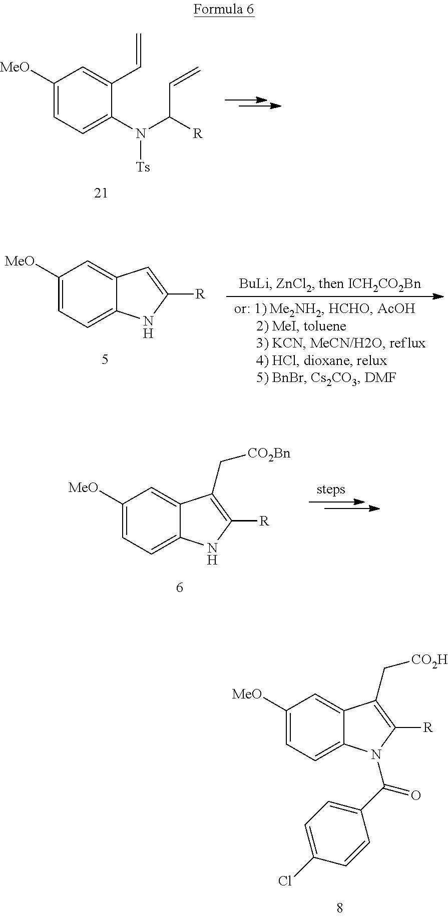

In summary, the existing problems in the prior art are as follows: most of the existing indomethacin synthesis methods are achieved by construction of an indole ring and modification; simple structural changes of an indomethacin molecule based on this synthetic strategy often require de novo synthesis; thus, the steps are relatively lengthy, and there are certain limitations of functional groups and substrates, which are not conducive to the structural modification of indomethacin molecules and further biological function research.

Difficulty in solving the above technical problems: Directly starting the indole ring per se for functionalization is obviously conducive to the synthesis of indomethacin analogues and the corresponding structure-activity relationship studies. However, how to selectively introduce the corresponding substituents at the C2 and C3 positions of indole while avoiding the use of redundant protecting groups is obviously a considerable challenge.

Significance in solving the above technical problems: A substituent may be directly selectively introduced at the C2 position of indole through a palladium-catalyzed reaction; subsequently, a carboxyl fragment is introduced at the C3 position of the indole through the oxidative coupling of enol and indole, followed by introducing an acyl group at the N1 position. Through such a synthetic strategy, the corresponding functional groups may be introduced at the C2, C3 and N1 positions of the indole in the absence of a protecting group or a directing group, which is very conducive to the establishment of a rapidly constructed indomethacin-like small molecule library, and provides a powerful tool for further biological function research.

### SUMMARY

In view of the problems existing in the prior art, the present disclosure provides an indomethacin, an analogue, a synthetic method and use thereof.

The present disclosure is achieved by a method for synthesizing an indomethacin and an analogue thereof, and the method for synthesizing an indomethacin and an analogue thereof includes steps of:

introducing an alkyl group, an aromatic ring or a heteroaromatic ring directly at the C2 position of indole, a carboxylic acid fragment at the C3 position of the indole, and an aroyl group at the N1 position of the indole through palladium-catalyzed reactions.

Further, the method for synthesizing an indomethacin and an analogue thereof may have the following synthetic reaction formula:

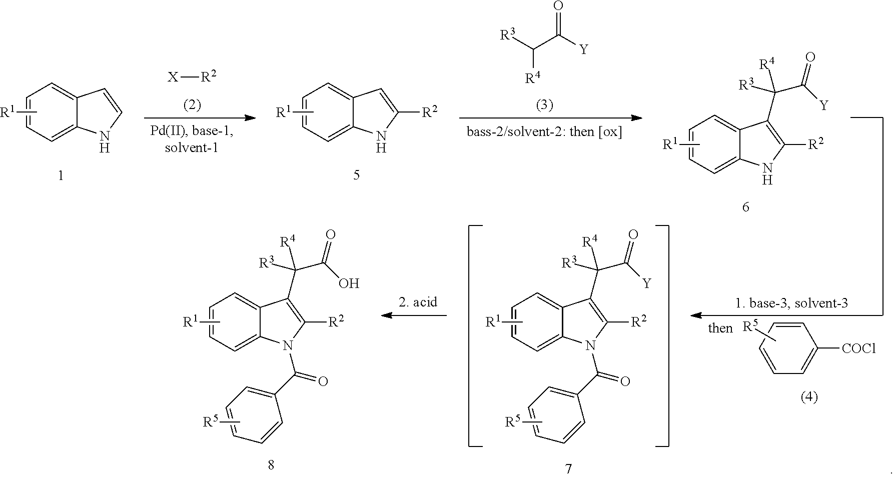

Further, in the method for synthesizing an indomethacin and an analogue thereof, R¹, R², R³, R⁴, and R⁵ may be either identical or different; R¹ may be selected from the group consisting of alkyl group, alkoxy group, halogen, ester group, amide, amino group, substituted aromatic or heteroaromatic ring at the C4, C5, C6 and C7 positions of an indole 1, either mono-substituted or poly-substituted; R² may be a fragment in a halogenated compound 2 and may be selected from the group consisting of alkyl group, aromatic and aromatic rings, and X may be selected from the group consisting of bromine, iodine, chlorine, and halogenoid functional groups from OTs, OMs, and OTf; R³ and R⁴ may be α-substituents of an acetate derivative 4, comprising alkyl groups, aromatic and aromatic rings, and Y may be selected from the group consisting of alkoxy group, amino group, amide, and sulfonamide; R⁵ may be a substituent on the aromatic ring of an aryl acyl halide 6 and may be selected from the group consisting of halogen, alkyl group, alkoxy group, amino group, hydroxyl group, nitro group, alkenyl group, sulfhydryl group, carboxyl group, ketone, sulfone, sulfoxide, carboxyl group, ester, amide, and phosphoramide at the C2, C3, C4, C5, and C6 positions.

Further, raw materials 1 to 4 used in the method for synthesizing an indomethacin and an analogue thereof may be as follows:

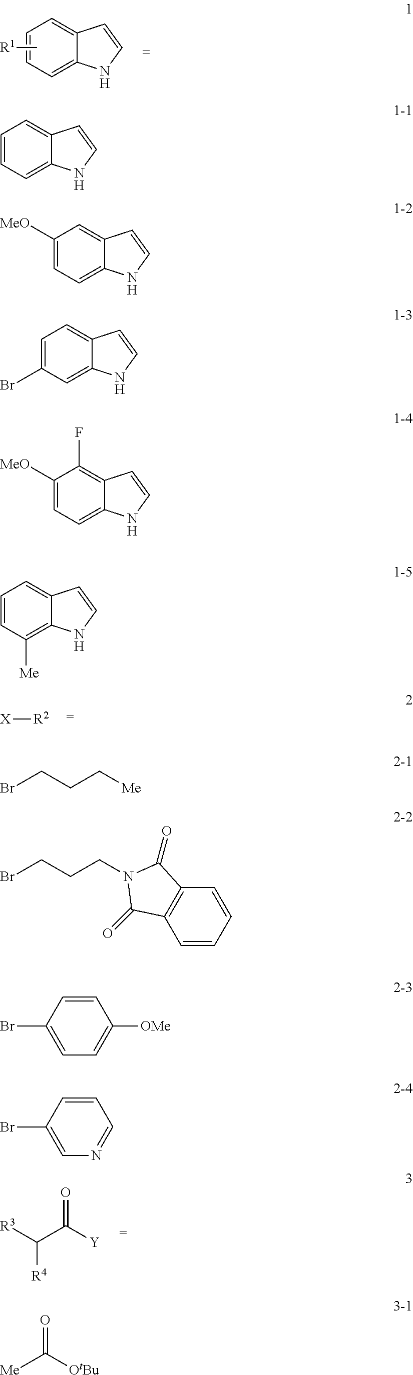

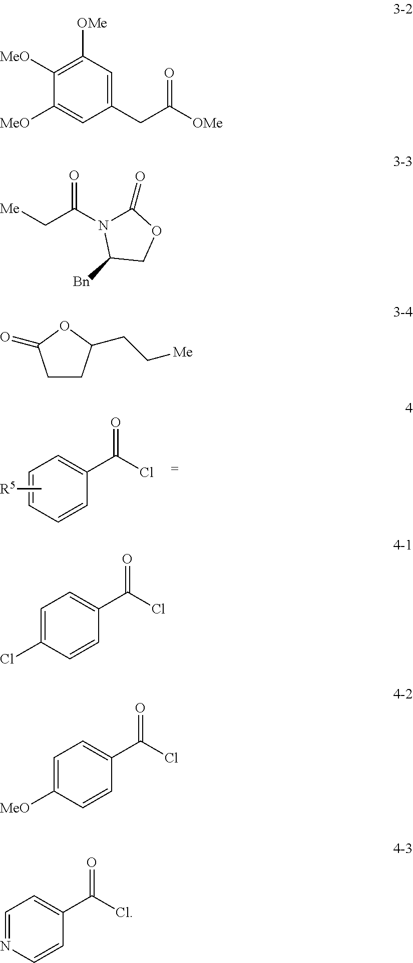

Further, intermediates 5 to 8 synthesized by the method for synthesizing an indomethacin and an analogue thereof may be as follows:

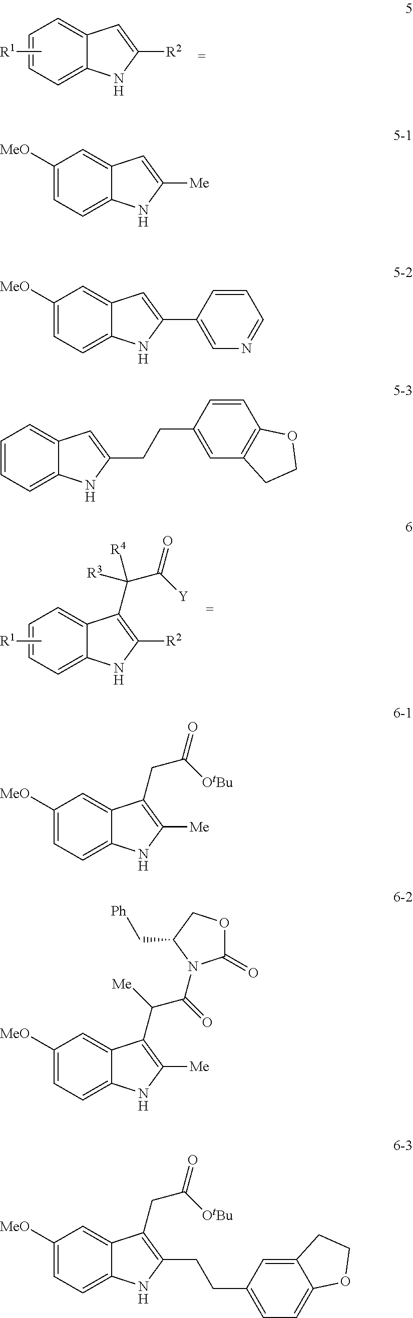

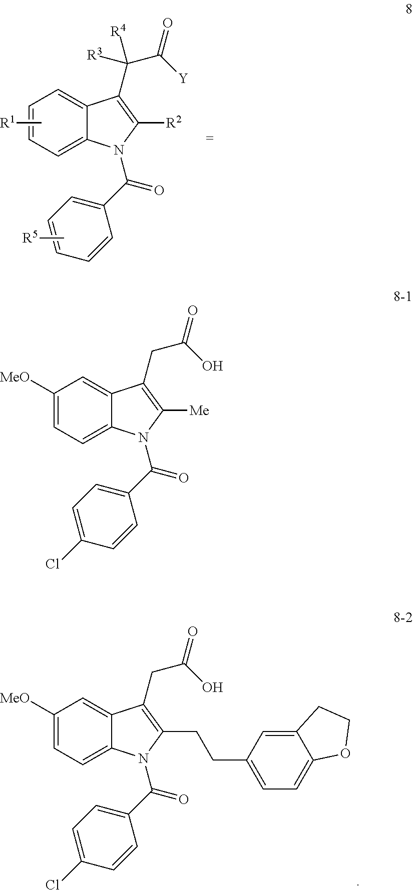

Further, the catalysts Pd(II) for the method for synthesizing an indomethacin and an analogue thereof may include PdCl₂, PdCl₂(MeCN)₂, Pd(OAc)₂, Pd(OTf)₂, Pd(acac)₂, and Pd(OTs)₂; the base-1 may include Na₂CO₃, K₂CO₃, Cs₂CO₃, Rb₂CO₃, NaHCO₃, KHCO₃, RbHCO₃, CsHCO₃, NaOAc, KOAc, RbOAc, CsOAc, K₃PO₄, Na₃PO₄, NaO^{t}Bu, NaOPiv, and KOPiv; the solvent-1 may include dimethyformamide (DMF), dimethylacetamide (DMA), toluene, N-methylpyrrolidone, tetrahydrofuran (THF), 2-methyltetrahydrofuran, dioxane, and glycol dimethyl ether; and the above catalyst, base and solvent may be mixed from one or more thereof;

the base-2 may include, but be not limited to, lithium diisopropylamide (LDA), LiHMDS, NaHMDS, KHMDS, K^{t}OBu, LiTMP, and NaH; the oxidant [ox] may include one or more of iodine, excessive iodine, oxygen, peroxide compounds, cupric salts, ferric salts, titanic salts, MnO₂, KMnO₄, ammonium molybdate, and ceric ammonium nitrate; the solvent-2 may include DMF, DMA, toluene, N-methylpyrrolidone, THF, 2-methyltetrahydrofuran, dioxane, and glycol dimethyl ether; and the above base, oxidant, and solvent may be mixed from one or more thereof;
the base-3 may include LDA, LiHMDS, NaHMDS, KHMDS, K^{t}OBu, NaO^{t}Bu, LiTMP, NaH, pyridine, trimethylamine (TEA), DBU, diisopropylethylamine (DIPEA), Na₂CO₃, and K₂CO₃; the solvent-3 may include DMF, DMA, toluene, N-methylpyrrolidone, THF, 2-methyltetrahydrofuran, dioxane, and glycol dimethyl ether; the acid may include trifluoroacetic acid (TFA), trichloroacetic acid (TCA), acetic acid, hydrochloric acid, sulfuric acid, and phosphonic acid; and the above base, solvent, and acid may be mixed from one or more thereof.

Further, in the method for synthesizing an indomethacin and an analogue thereof, compounds 1 and 2 may have a ratio of 5:1 to 1:5; the amount of the catalyst Pd(II) may be 0.5-50%; the amount of the base may be 1-6 equivalents, reaction time may be 2-48 h, and reaction temperature may be 20-150° C.;

compounds 5 and 3 have a ratio of 5:1 to 1:5; the amount of the base is 1-6 equivalents, the amount of the oxidant is 1-6 equivalents, the reaction time is from 15 min to 24 h, and the reaction temperature is −78-80° C.;
compounds 6 and 4 have a ratio of 5:1 to 1:5; the amount of the base is 1-10 equivalents, the amount of the acid is 1-20 equivalents, the reaction time is 2-48 h, and the reaction temperature is −78-80° C.

Another objective of the present disclosure is to provide an indomethacin synthesized by the method for synthesizing an indomethacin and an analogue thereof. Indomethacin is an NSAID that is commonly used for anti-inflammation, analgesia, and antipyresis.

Still another objective of the present disclosure is to provide an indomethacin analogue synthesized by the method for synthesizing an indomethacin and an analogue thereof.

Indomethacin analogues have been reported to reverse tumor drug resistance, and the present disclosure may provide rapid construction of such compounds.

The present disclosure solves a problem: most of the existing indomethacin synthesis methods are achieved by construction of an indole ring and modification; simple structural changes of an indomethacin molecule based on this synthetic strategy often require de novo synthesis; the late modification and structure-activity relationship study of the indomethacin molecule have long synthetic steps.

### BRIEF DESCRIPTION OF THE DRAWINGS

FIG. 1 is a flow chart of a method for synthesizing an indomethacin and an analogue thereof provided by the examples of the present disclosure.

FIG. 2 illustrates the synthetic principle of indomethacin and analogues thereof provided by the examples of the present disclosure.

### DETAILED DESCRIPTION

In order to make the objectives, technical solutions, and advantages of the present disclosure clearer, the present disclosure will be further described in detail below in conjunction with examples. It should be understood that the specific examples described herein are merely intended to explain but not to limit the present disclosure.

In view of the problems existing in the prior art, the present disclosure provides an indomethacin, an analogue, a synthetic method and use thereof, and the present disclosure will be further described in detail below in conjunction with drawings.

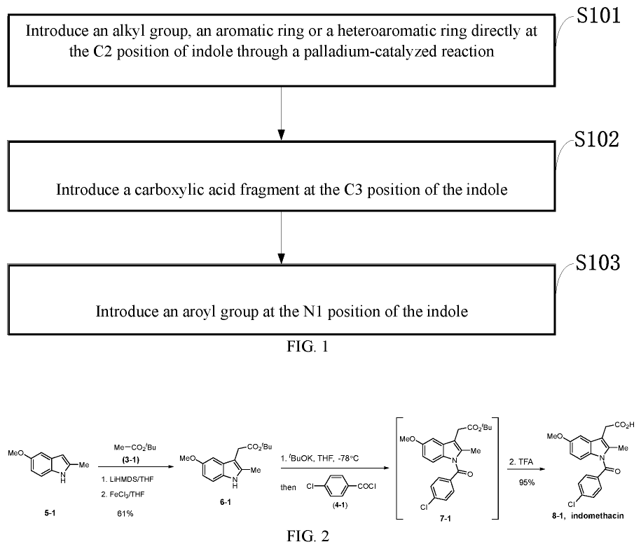

As shown in FIG. 1, the method for synthesizing an indomethacin and an analogue thereof provided by the examples of the present disclosure includes the following steps:

S101: introducing an alkyl group, an aromatic ring or a heteroaromatic ring directly at the C2 position of indole through a palladium-catalyzed reaction;
S102: introducing a carboxylic acid fragment at the C3 position of the indole; and
S103: introducing an aroyl group at the N1 position of the indole.

The method for synthesizing an indomethacin and an analogue thereof provided by the examples of the present disclosure implements a three-step method for synthesizing an indomethacin and an analogue thereof starting from an indole molecule (Formula 6).

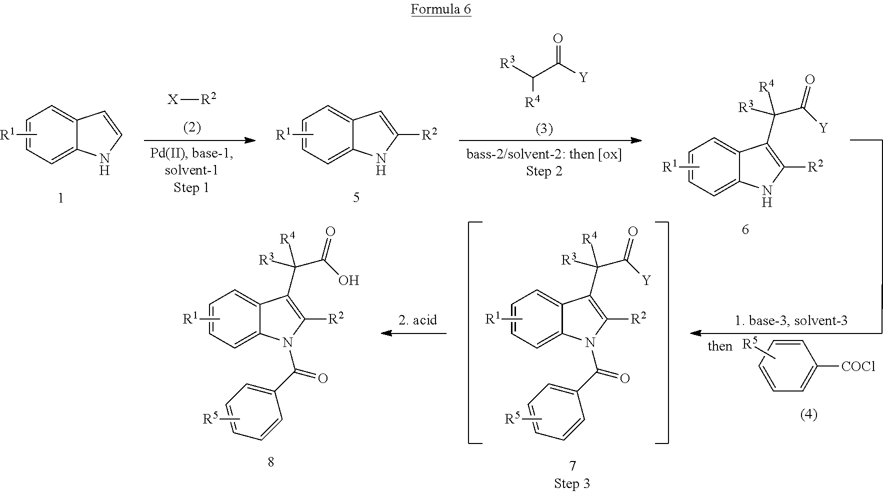

In a preferred example of the present disclosure, R¹, R², R³, R⁴, and R⁵ may be either identical or different; R¹ may be selected from the group consisting of alkyl group, alkoxy group, halogen, ester group, amide, amino group, substituted aromatic or heteroaromatic ring at the C4, C5, C6 and C7 positions of an indole 1, either mono-substituted or poly-substituted; R² may be a fragment in a halogenated compound 2 and may be selected from the group consisting of alkyl group, aromatic and aromatic rings, and X may be selected from the group consisting of bromine, iodine, chlorine, and halogenoid functional groups from OTs, OMs, and OTf; R³ and R⁴ may be α-substituents of an acetate derivative 4, comprising alkyl groups, aromatic and aromatic rings, and Y may be selected from the group consisting of alkoxy group, amino group, amide, and sulfonamide; R⁵ may be a substituent on the aromatic ring of an aryl acyl halide 6 and may be selected from the group consisting of halogen, alkyl group, alkoxy group, amino group, hydroxyl group, nitro group, alkenyl group, sulfhydryl group, carboxyl group, ketone, sulfone, sulfoxide, carboxyl group, ester, amide, and phosphoramide at the C2, C3, C4, C5, and C6 positions.

In a preferred example of the present disclosure, raw materials 1 to 4 used may be specifically as the following examples, but may not be limited thereto (Formula 7):

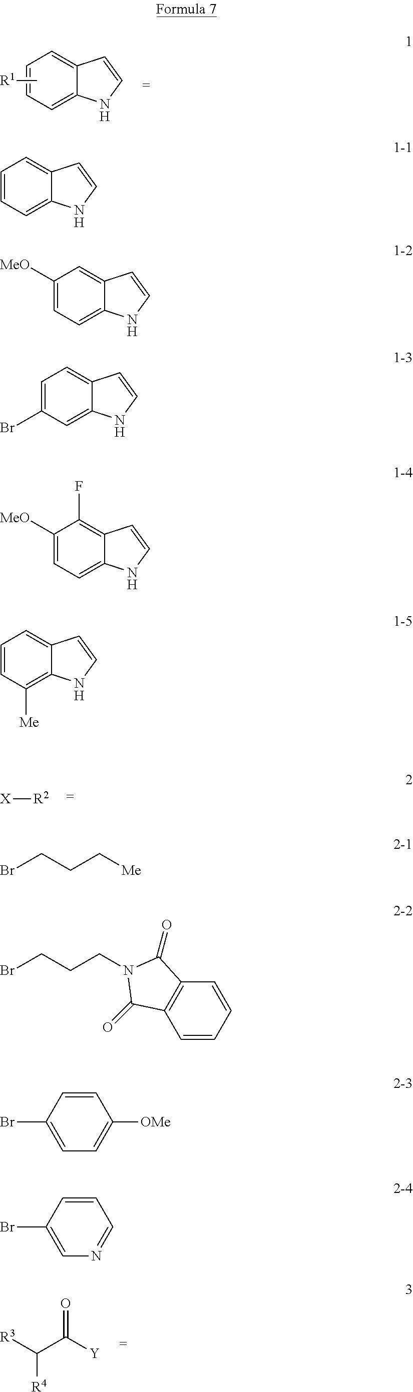

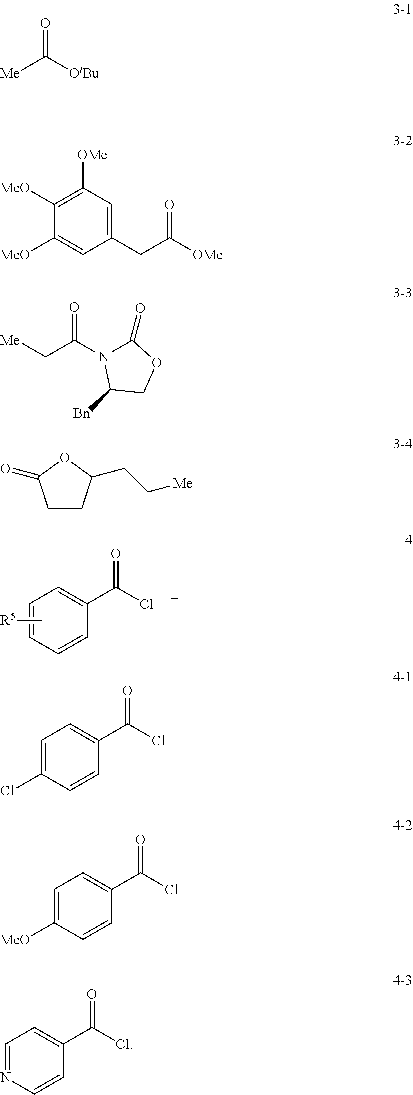

In a preferred example of the present disclosure, in a synthetic route, synthesized intermediates 5 to 8 of the indomethacin and the analogue thereof may be typical but not limited to the following (Formula 8).

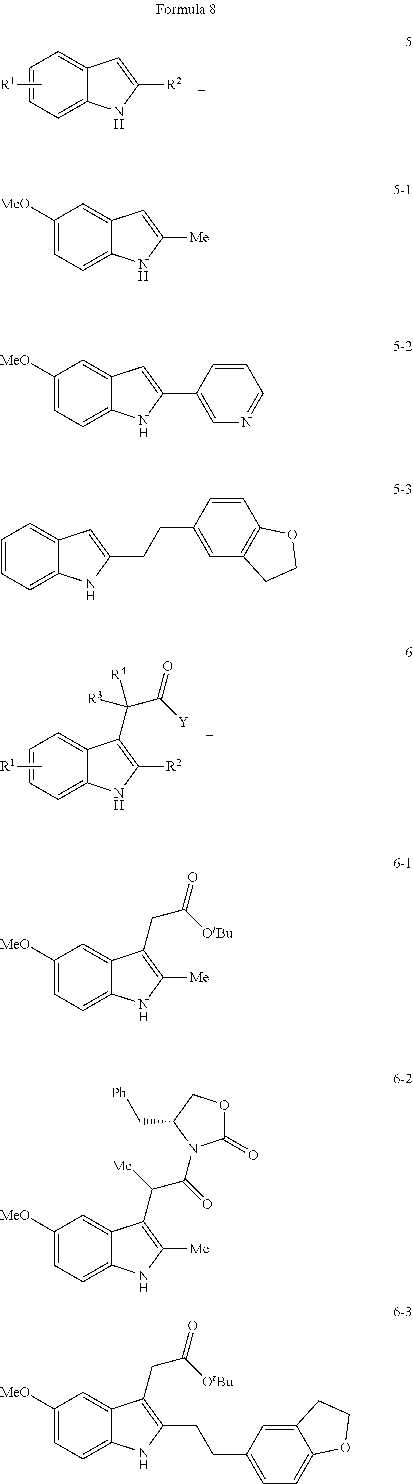

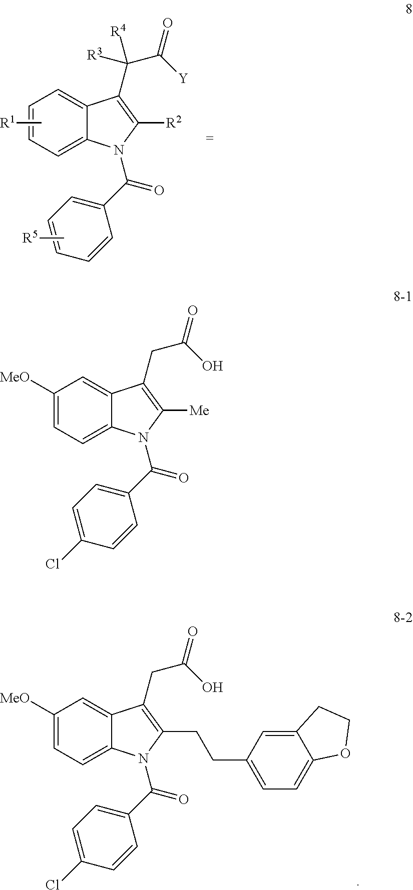

In a preferred example of the present disclosure, the catalyst Pd(II) described in step 1 of Formula 6 may include, but not be limited to, PdCl₂, PdCl₂(MeCN)₂, Pd(OAc)₂, Pd(OTf)₂, Pd(acac)₂, and Pd(OTs)₂; the base-1 may include, but not be limited to, Na₂CO₃, K₂CO₃, Cs₂CO₃, Rb₂CO₃, NaHCO₃, KHCO₃, RbHCO₃, CsHCO₃, NaOAc, KOAc, RbOAc, CsOAc, K₃PO₄, Na₃PO₄, NaO^{t}Bu, NaOPiv, and KOPiv; the solvent-1 may include, but not be limited to, DMF, DMA, toluene, N-methylpyrrolidone, THF, 2-methyltetrahydrofuran, dioxane, and glycol dimethyl ether; and the above catalyst, base and solvent may be mixed from one or more thereof.

In a preferred example of the present disclosure, the base-2 in step 2 of Formula 6 may include, but be not limited to, LDA, LiHMDS, NaHMDS, KHMDS, K^{t}OBu, LiTMP, and NaH; the oxidant [ox] may include, but be not limited to, one or more of iodine, excessive iodine, oxygen, peroxide compounds, cupric salts (such as CuCl₂ and Cu(OAc)₂), ferric salts (such as FeCl₃ and Fe(acac)₃), titanic salts (such as TiCl₄), MnO₂, KMnO₄, ammonium molybdate, and ceric ammonium nitrate; the solvent-2 may include, but be not limited to, DMF, DMA, toluene, N-methylpyrrolidone, THF, 2-methyltetrahydrofuran, dioxane, and glycol dimethyl ether; and the above base, oxidant, and solvent may be mixed from one or more thereof.

In a preferred example of the present disclosure, the base-3 in step 3 of Formula 6 may include, but be not limited to, LDA, LiHMDS, NaHMDS, KHMDS, K^{t}OBu, NaO^{t}Bu, LiTMP, NaH, pyridine, TEA, DBU, DIPEA, Na₂CO₃, and K₂CO₃; the solvent-3 may include, but be not limited to, DMF, DMA, toluene, N-methylpyrrolidone, THF, 2-methyltetrahydrofuran, dioxane, and glycol dimethyl ether; the acid may include, but be not limited to, TFA, TCA, acetic acid, hydrochloric acid, sulfuric acid, and phosphonic acid; and the above base, solvent, and acid may be mixed from one or more thereof.

In a preferred example of the present disclosure, in step 1 of Formula 6, compounds 1 and 2 may have a ratio of 5:1 to 1:5; the amount of the catalyst Pd(II) may be 0.5-50%; the amount of the base may be 1-6 equivalents, reaction time may be 2-48 h, and reaction temperature may be 20-150° C.

In a preferred example of the present disclosure, in step 2 of Formula 6, compounds 5 and 3 may have a ratio of 5:1 to 1:5; the amount of the base may be 1-6 equivalents (relative to the raw material least-used in the reaction), the amount of the oxidant may be 1-6 equivalents, the reaction time may be from 15 min to 24 h, and the reaction temperature may be −78-80° C.

In a preferred example of the present disclosure, in step 3 of Formula 6, compounds 6 and 4 may have a ratio of 5:1 to 1:5; the amount of the base may be 1-10 equivalents (relative to the raw material least-used in the reaction), the amount of the acid may be 1-20 equivalents, the reaction time may be from 2-48 h, and the reaction temperature may be −78-80° C.

The technical solutions of the present disclosure will be further described below in conjunction with specific examples.

### Example 1: Indomethacin Synthesis (Formula 9)

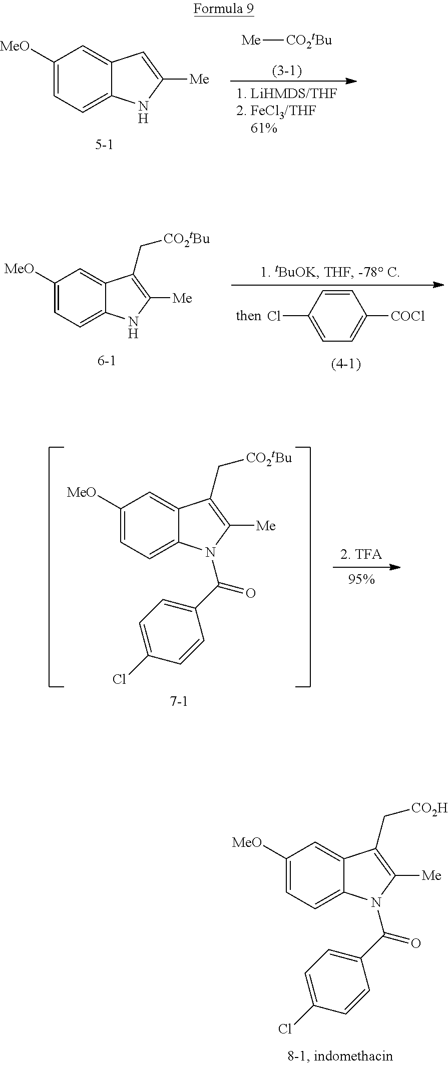

At room temperature, compounds 5-1 (161 mg, 1.0 mmol, 2.0 equiv.) and 3-1 (58 mg, 0.5 mmol, 2.0 equiv.) were dissolved in anhydrous THF and cooled to −78° C., and LiHMDS (1.3 M in THF, 1.54 mL, 2.0 mmol, 4.0 equiv.) was added dropwise under nitrogen. After a reaction solution was stirred for 0.5 h at −78° C., FeCl₃ (322 mg, 2.0 mmol, 4.0 equiv.) was added. After reacting for 2 h at −78° C., water was added to quench the reaction, and the temperature was raised to room temperature and ethyl acetate was added, followed by washing with water and saturated brine successively and drying over anhydrous sodium sulfate. After filtration, concentration, and column chromatography, a desired product 6-1 (84.3 mg, yield 61%) was obtained.

The compound 6-1 (15 mg, 0.054 mmol, 1.0 equiv.) was added to a 20 mL reaction tube at one time; 4 mL of anhydrous THF was added under nitrogen and stirred for a while at room temperature; the mixture was cooled to −78° C. after all the raw materials were dissolved. Subsequently, KO^{t}Bu (1.0 M in THF, 0.076 mL, 1.4 equiv.) was added dropwise, and the reaction was conducted at this temperature while stirring for 1 h; 4-chlorobenzoyl chloride (xx mg, xx mmol, 1.3 equiv.) was added. After the reaction was monitored by a TLC plate, NH₄Cl (aq., 15 mL) was added to quench, followed by diluting with 50 mL of ethyl acetate, and washing with water (15 mL) and saturated brine (15 mL). Organic phase was dried over anhydrous sodium sulfate. After a while, sodium sulfate was filtered off, and the solvent was removed on a rotary evaporator; next, a desired intermediate 7-1 (21.6 mg, yield 96%) was separated by silica gel column chromatography. The intermediate 7-1 was dissolved in excess TFA (1 mL). After the reaction was monitored by TLC, TFA was removed on the rotary evaporator, and a crude product was separated by column chromatography to obtain an indomethacin 8-1 (18.4 mg, yield 99%).

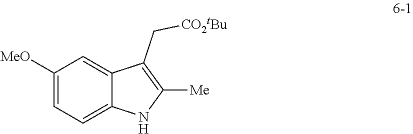 [SMILES: COc1ccc2[nH]c(C)c(CC(=O)OC(C)(C)C)c2c1]

Brown solid, 84.3 mg, yield 61% (0.5 mmol scale)

¹H NMR (400 MHz, CDCl₃) δ 7.85 (s, 1H), 7.08 (d, J=8.7 Hz, 1H), 7.01 (d, J=2.5 Hz, 1H), 6.76 (dd, J=8.7, 2.4 Hz, 1H), 3.86 (s, 3H), 3.56 (s, 2H), 2.32 (s, 3H), and 1.44 (s, 9H).

¹³C NMR (101 MHz, CDCl₃) δ 171.7, 154.0, 133.6, 130.4, 129.1, 111.0, 110.9, 105.1, 100.6, 80.6, 56.0, 32.0, 28.2 (×3C), and 11.8.

HRMS-API (m/z): calcd. for C₁₆H₂₂NO₃ [M+H⁺] 276.1594, found 276.1595.

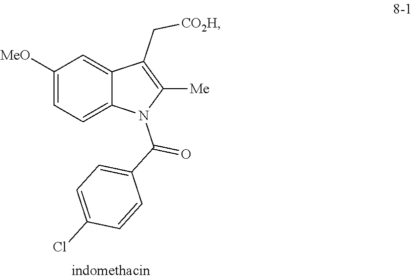 [SMILES: COc1ccc2c(c1)c(CC(=O)O)c(C)n2C(=O)c1ccc(Cl)cc1]

White solid, 18.4 mg, yield 95%

¹H NMR (400 MHz, CDCl₃) δ 7.70-7.61 (m, 2H), 7.47 (d, J=8.4 Hz, 2H), 6.95 (d, J=2.5 Hz, 1H), 6.85 (d, J=9.0 Hz, 1H), 6.67 (dd, J=9.0, 2.5 Hz, 1H), 3.83 (s, 3H), 3.69 (s, 2H), and 2.39 (s, 3H).

### Example 2: Synthesis of Indomethacin Analogue 7-2 (Formula 10)

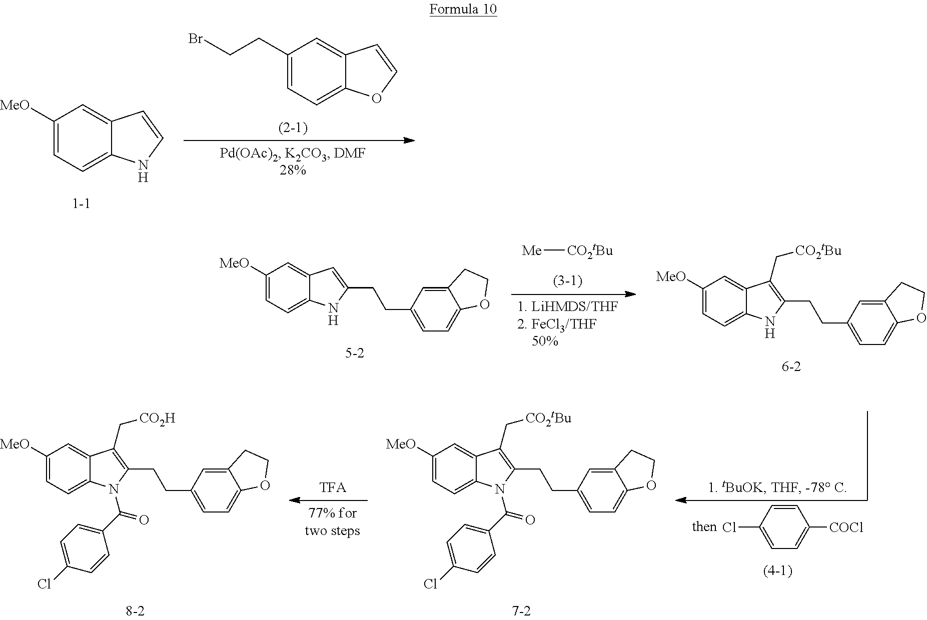

Indole 1-1 (1.75 g, 10 mmol, 1.0 equiv.), halogenated hydrocarbon 2-1 (xx mg, 10 mmol, 1.0 equiv.), Pd(OAc)₂ (224 mg, 1.0 mmol, 1.0 equiv.), norbornene (1.88 g, 20 mmol, 2.0 equiv.) and K₂CO₃ (2.76 g, 20 mmol, 2.0 equiv.) were added in a 100 mL round bottom flask. Subsequently, DMA (0.5 M H₂O) was added under nitrogen, heated to 80° C. and stirred for 18 h. After the reaction was completed, 100 mL of dichloromethane was added for dilution, and insolubles were removed by filtration. The solution was washed with water and saturated brine to remove DMA, and dried over Na₂SO₄. The solvent was removed by filtration, and a crude product was purified by silica gel chromatography to obtain a desired compound 5-2 (723 mg, yield 28%).

Compounds 6-2, 7-2 and 8-2 were synthesized in the same way as compounds 6-1, 6-2 and 7-2.

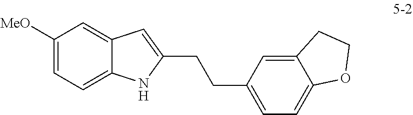 [SMILES: COc1ccc2[nH]c(CCc3ccc4c(c3)CCO4)cc2c1]

Yellow solid, 732.3 mg, yield 28% (10 mmol scale)

¹H NMR (400 MHz, CDCl₃) δ 7.78 (s, 1H), 7.15 (d, J=8.7 Hz, 1H), 7.06 (t, J=2.6 Hz, 2H), 6.96 (dd, J=8.2, 1.9 Hz, 1H), 6.82 (dd, J=8.7, 2.5 Hz, 1H), 6.76 (dd, J=8.1, 1.8 Hz, 1H), 6.23 (d, J=2.2 Hz, 1H), 4.58 (t, J=8.7 Hz, 2H), 3.87 (d, J=1.6 Hz, 3H), 3.19 (t, J=8.6 Hz, 2H), and 3.04-2.93 (m, 4H).

¹³C NMR (101 MHz, CDCl₃) δ 158.5, 154.1, 140.2, 133.3, 131.0, 129.2, 127.8, 127.3, 125.0, 111.1, 110.9, 109.1, 102.1, 99.6, 71.3, 55.9, 35.1, 30.7, and 29.8.

HRMS-API (m/z): calcd. for C₁₉H₁₈NO₂ [M+H⁺] 292.1332, found 292.1339.

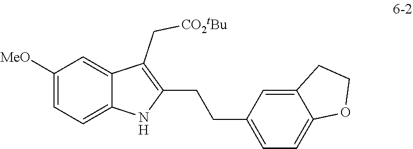 [SMILES: COc1ccc2[nH]c(CCc3ccc4c(c3)CCO4)c(CC(=O)OC(C)(C)C)c2c1]

Yellow solid, 102.2 mg, yield 50% (0.5 mmol scale)

¹H NMR (400 MHz, CDCl₃) δ 7.76 (s, 1H), 7.12-7.03 (m, 2H), 6.97 (d, J=1.8 Hz, 1H), 6.88 (dd, J=8.1, 1.9 Hz, 1H), 6.77 (dd, J=8.7, 2.5 Hz, 1H), 6.71 (d, J=8.1 Hz, 1H), 4.56 (t, J=8.7 Hz, 2H), 3.87 (s, 3H), 3.54 (s, 2H), 3.15 (t, J=8.6 Hz, 2H), 2.97 (t, J=7.1 Hz, 2H), 2.87 (t, J=7.4 Hz, 2H), and 1.45 (s, 9H).

¹³C NMR (101 MHz, CDCl₃) δ 171.6, 158.6, 154.0, 137.3, 133.3, 130.3, 128.9, 127.9, 127.2, 125.1, 111.1, 109.1, 105.0, 100.7, 80.6, 71.3, 55.9, 35.6, 32.0, 29.8, 28.9, 28.2, and 28.2 (×3C).

HRMS-API (m/z): calcd. for C₂₅H₃₀NO₄ [M+H⁺] 408.2169, found 408.2169.

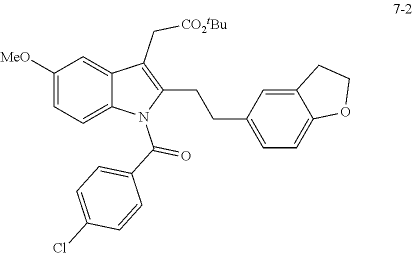 [SMILES: COc1ccc2c(c1)c(CC(=O)OC(C)(C)C)c(CCc1ccc3c(c1)CCO3)n2C(=O)c1ccc(Cl)cc1]

Yellow oil, 69.9 mg, yield 81% (0.158 mmol scale,)

¹H NMR (400 MHz, CDCl₃) δ 7.63-7.54 (m, 2H), 7.51-7.41 (m, 2H), 7.00 (dd, J=4.5, 2.1 Hz, 2H), 6.86 (dd, J=8.0, 1.9 Hz, 1H), 6.67-6.58 (m, 2H), 6.55 (d, J=9.0 Hz, 1H), 4.48 (t, J=8.7 Hz, 2H), 3.83 (s, 3H), 3.54 (s, 2H), 3.23 (dd, J=9.4, 6.3 Hz, 2H), 3.06 (t, J=8.7 Hz, 2H), 2.84 (dd, J=9.1, 6.5 Hz, 2H), and 1.47 (s, 9H).

¹³C NMR (101 MHz, CDCl₃) δ 170.3, 168.4, 158.5, 155.9, 140.0, 139.5, 133.7, 133.1, 131.4 (×2C), 131.0, 130.7, 129.1 (×2C), 128.1, 127.2, 125.1, 114.9, 113.6, 111.8, 109.0, 101.6, 81.2, 71.2, 55.7, 35.9, 31.8, 29.8, 28.6, and 28.2 (×3C).

HRMS-API (m/z): calcd. for C₃₂H₃₃NO₅ [M+H⁺] 546.2041, found 546.2033.

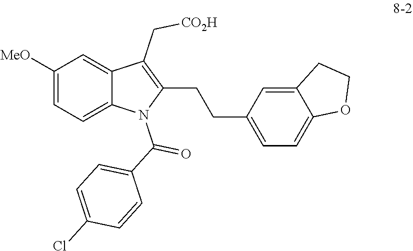 [SMILES: COc1ccc2c(c1)c(CC(=O)O)c(CCc1ccc3c(c1)CCO3)n2C(=O)c1ccc(Cl)cc1]

White solid, 17.5 mg, yield 77%

¹H NMR (400 MHz, CDCl₃) δ 7.60 (d, J=8.2 Hz, 2H), 7.45 (d, J=8.2 Hz, 2H), 6.95 (d, J=3.0 Hz, 2H), 6.80 (d, J=8.0 Hz, 1H), 6.61 (td, J=6.9, 3.1 Hz, 2H), 6.53 (d, J=9.0 Hz, 1H), 4.47 (t, J=8.6 Hz, 2H), 3.80 (s, 3H), 3.61 (s, 2H), 3.23 (t, J=7.7 Hz, 2H), 3.04 (t, J=8.6 Hz, 2H), and 2.81 (t, J=7.7 Hz, 2H).

¹³C NMR (101 MHz, CDCl₃) δ 176.7, 168.5, 158.6, 156.0, 140.4, 139.7, 133.6, 132.9, 131.5 (×2C), 131.0, 130.4, 129.2 (×2C), 128.1, 127.3, 125.1, 115.0, 112.3, 111.9, 109.1, 101.6, 71.2, 55.8, 35.7, 30.1, 29.8, and 28.7.

HRMS-API (m/z): calcd. for C₃₂H₃₃ClNO₅ [M+H⁺] 546.2041, found 546.2033.

The above descriptions are merely preferred examples of the present disclosure, and are not intended to limit the present disclosure. Any modification, equivalent substitute and improvement without departing from the spirit and principle of the present disclosure shall be included within the protection scope of the present disclosure.

1. A method for synthesizing an indomethacin and analogues thereof, wherein the method comprises the steps of:
introducing an alkyl group, an aromatic ring or a heteroaromatic ring directly at the C2 position of an indole, introducing a carboxylic acid fragment at the C3 position of the indole, and introducing an aroyl group at the N1 position of the indole through palladium-catalyzed reactions.

2. The method for synthesizing an indomethacin and analogues thereof according to claim 1, wherein the method proceeds according to the following reaction steps and wherein 1 is the starting indole; 5, 6 and 7 are chemical intermediates; 8 is the end product and (2), (3) and (4) (parts 1 and 2) are chemical reaction steps using chemical reagents as shown:

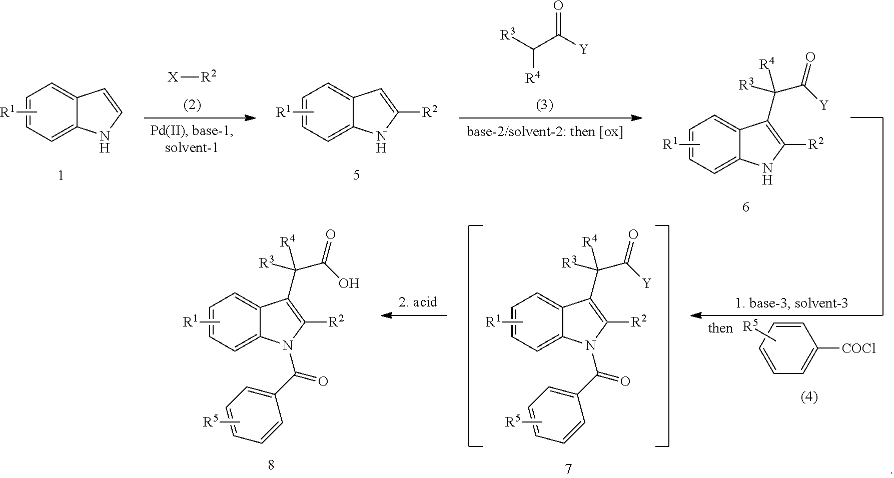

3. The method for synthesizing an indomethacin and analogues thereof according to claim 2, wherein in the chemical formulas: R¹, R², R³, R⁴, and R⁵ are either identical or different; R¹ is selected from the group consisting of alkyl group, alkoxy group, halogen, ester group, amide, amino group, a substituted aromatic or heteroaromatic ring at one or more of the C4, C5, C6 and C7 positions of the indole 1, either mono-substituted or poly-substituted; R² is a fragment in a halogenated compound used in reaction (2) and is selected from the group consisting of alkyl group, aromatic and heteroaromatic rings, and X is selected from the group consisting of bromine, iodine, chlorine, and halogenoid functional groups from OTs, OMs, and OTf; R³ and R⁴ are α-substituents of an acetate derivative used in reaction (3), comprising alkyl groups, aromatic and heteroaromatic rings, and Y is selected from the group consisting of alkoxy group, amino group, amide, and sulfonamide; and R⁵ is a substituent on the aromatic ring of an aryl acyl halide used in reaction (4) and is selected from the group consisting of halogen, alkyl group, alkoxy group, amino group, hydroxyl group, nitro group, alkenyl group, sulfhydryl group, carboxyl group, ketone, sulfone, sulfoxide, ester, amide, and phosphoramide at one or more of the C2, C3, C4, C5, and C6 positions.

4. The method for synthesizing an indomethacin and analogues thereof according to claim 2, wherein the raw materials numbered 1 to 4 below and variants thereof used in synthesizing an indomethacin and analogues thereof are as follows:

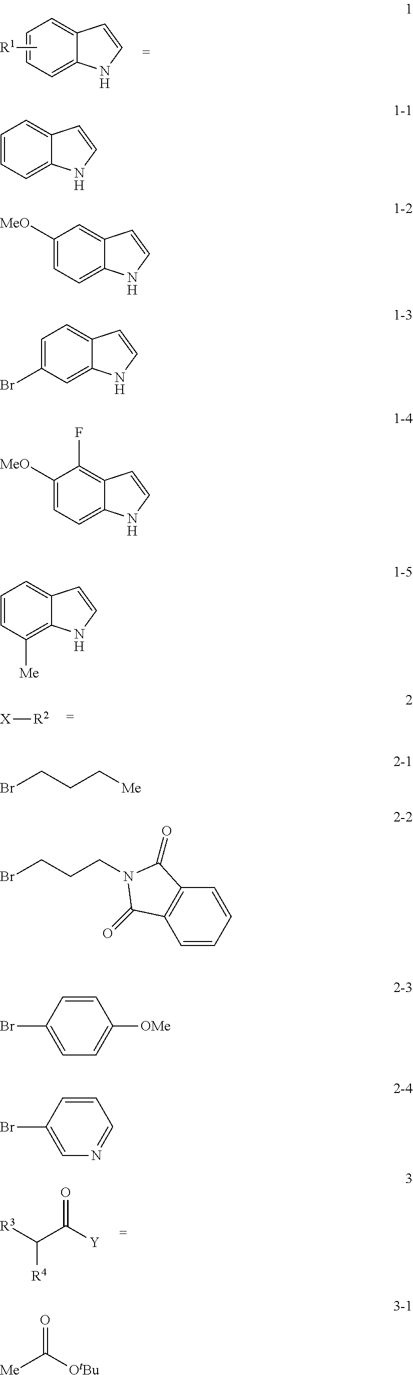

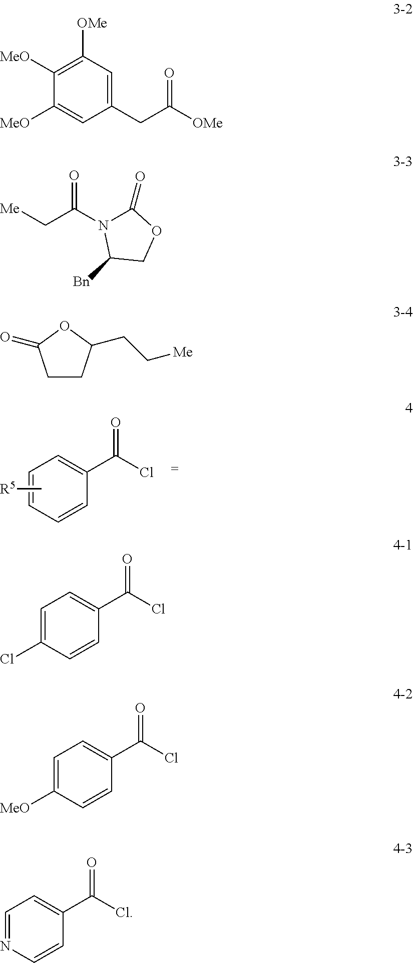

5. The method for synthesizing an indomethacin and analogues thereof according to claim 2, wherein the intermediates numbered 5 and 6 and the product 8 below and variants thereof synthesized by the method are as follows:

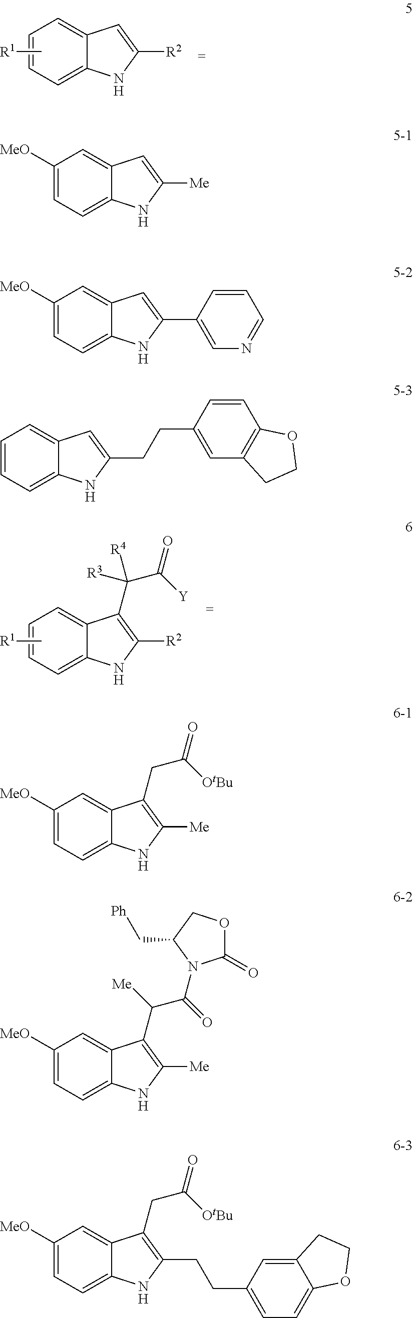

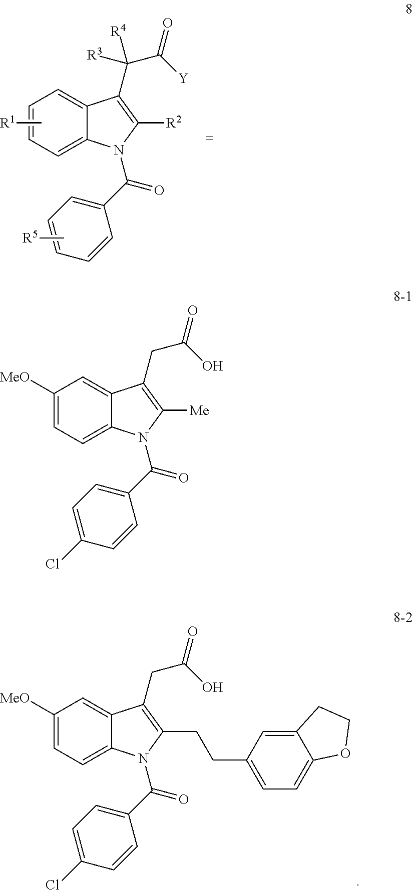

6. The method for synthesizing an indomethacin and analogues thereof according to claim 2, wherein: the palladium catalysts Pd(II) used in chemical reaction step (2) are selected from the group consisting of PdCl₂, PdCl₂(MeCN)₂, Pd(OAc)₂, Pd(OTf)₂, Pd(acac)₂, and Pd(OTs)₂; the base-1 used in chemical reaction step (2) is selected from the group consisting of Na₂CO₃, K₂CO₃, Cs₂CO₃, Rb₂CO₃, NaHCO₃, KHCO₃, RbHCO₃, CsHCO₃, NaOAc, KOAc, RbOAc, CsOAc, K₃PO₄, Na₃PO₄, NaO^{t}Bu, NaOPiv, and KOPiv; the solvent-1 used in chemical reaction step (2) is selected from the group consisting of dimethyformamide (DMF), dimethylacetamide (DMA), toluene, N-methylpyrrolidone, tetrahydrofuran (THF), 2-methyltetrahydrofuran, dioxane, and glycol dimethyl ether; and further wherein the above catalyst, base and solvent are mixed from one or more thereof;
the base-2 used in chemical reaction step (3) comprises but is not limited to lithium diisopropylamide (LDA), LiHMDS, NaHMDS, KHMDS, K^{t}OBu, LiTMP, and NaH; the oxidant [ox] used in chemical reaction step (3) comprises one or more of iodine, excessive iodine, oxygen, peroxide compounds, cupric salts, ferric salts, titanic salts, MnO₂, KMnO₄, ammonium molybdate, and ceric ammonium nitrate; the solvent-2 used in chemical reaction step (3) comprises DMF, DMA, toluene, N-methylpyrrolidone, THF, 2-methyltetrahydrofuran, dioxane, and glycol dimethyl ether; and further wherein the above base, oxidant, and solvent are mixed from one or more thereof;
the base-3 used in the first part of chemical reaction step (4) comprises LDA, LiHMDS, NaHMDS, KHMDS, K^{t}OBu, NaO^{t}Bu, LiTMP, NaH, pyridine, trimethylamine (TEA), DBU, diisopropylethylamine (DIPEA), Na₂CO₃, and K₂CO₃; the solvent-3 used in the first part of chemical reaction step (4) comprises DMF, DMA, toluene, N-methylpyrrolidone, THF, 2-methyltetrahydrofuran, dioxane, and glycol dimethyl ether; and the acid used in the second part of chemical reaction step (4) comprises trifluoroacetic acid (TFA), trichloroacetic acid (TCA), acetic acid, hydrochloric acid, sulfuric acid, and phosphonic acid; and further wherein the above base, solvent, and acid are mixed from one or more thereof.

7. The method for synthesizing an indomethacin and analogues thereof according to claim 2, wherein: in chemical reaction step (2), compound 1 and the halogenated compound used in chemical reaction step (2) have a molar ratio ranging from 5:1 to 1:5; the amount of the catalyst Pd(II) is 0.5-50%; the amount of the base-1 is 1-6 equivalents; reaction time is 2-48 h; and reaction temperature is 20-150° C.;
in chemical reaction step (3), compound 5 and the acetate derivative used in chemical reaction step (3) have a molar ratio ranging from 5:1 to 1:5; the amount of the base-2 is 1-6 equivalents; the amount of the oxidant is 1-6 equivalents; the reaction time is from 15 min to 24 h; and the reaction temperature is −78-80° C.; and
in chemical reaction step (4), compound 6 and the aryl acyl halide used in chemical reaction step (4) have a molar ratio ranging from 5:1 to 1:5; the amount of the base-3 is 1-10 equivalents; the amount of the acid is 1-20 equivalents; the reaction time is 2-48 h; and the reaction temperature is −78-80° C.

8. An indomethacin and analogues thereof synthesized by the method according claim 1.

9. An indomethacin and analogues thereof synthesized by the method according to claim 2.

10. (canceled)

11. An indomethacin and analogues thereof synthesized by the method according to claim 3.

12. An indomethacin and analogues thereof synthesized by the method according to claim 4.

13. An indomethacin and analogues thereof synthesized by the method according to claim 5.

14. An indomethacin and analogues thereof synthesized by the method according to claim 6.

15. An indomethacin and analogues thereof synthesized by the method according to claim 7.
